(19) United States
(12) Patent Application Publication
(10) Pub. No.: US 2023/0104606 A1
(43) Pub. Date: Apr. 6, 2023

(54) APPARATUSES AND METHODS TO FACILITATE PROOF OF AN IDENTITY OR ROLE

(71) Applicants: AT&T Intellectual Property I, L.P., Atlanta, GA (US); AT&T Intellectual Property II, L.P., Atlanta, GA (US)

(72) Inventors: Lars Benjamin Johnson, Brooklyn, NY (US); Umayal Roja Swarnam, Aurora, IL (US)

(73) Assignees: AT&T Intellectual Property I, L.P., Atlanta, GA (US); AT&T Intellectual Property II, L.P., Atlanta, GA (US)

(21) Appl. No.: 17/492,792

(22) Filed: Oct. 4, 2021

(51) Int. Cl. G16H 80/00; H04L 29/06; G16H 40/20; G16H 10/60

(52) U.S. Cl. CPC G16H 80/00; H04L 63/0428; G16H 40/20; G16H 10/60

(57) ABSTRACT

Aspects of the subject disclosure may include, for example, transmitting, by a processing system including a processor, a request for attention, wherein the processing system stores first data pertaining to a first user, receiving, by the processing system and based on the transmitting of the request for attention, a key from a second processing system including a second processor, based on the receiving of the key, identifying, by the processing system, a first portion of the first data to be transmitting to the second processing system, wherein the first portion of the first data is less than an entirety of the first data, and based on the identifying of the first portion of the first data, transmitting, by the processing system, the first portion of the first data to the second processing system. Other embodiments are disclosed.

### FIELD OF THE DISCLOSURE

The subject disclosure relates to apparatuses and methods to facilitate proof of an identity or role.

### BACKGROUND

As the world increasingly becomes connected via vast communication networks and via various communication devices, additional opportunities are generated to provision data that can be used to support various users/people. For example, having access to data in a medical context can prove to be beneficial, as enhanced patient care can be provided if, for example, knowledge of the patient's medical history/records can be obtained via the data. This may be particularly true in emergency situations, where an ability for first responders to obtain access to the data quickly may be a major determinant in realizing/obtaining positive outcomes/resolutions.

As the foregoing demonstrates, in many instances access to data can prove beneficial. However, access to patient-related data is accompanied by concerns for user/patient privacy/security in respect of such data. Many jurisdictions have enacted laws or regulations pertaining to medical data/records in terms of maintaining patient privacy/security. If a patient is unresponsive during an emergency, the question naturally arises whether, and to what extent, a first responder should have access to the patient's medical history/records. Even if the patient is responsive/alert during the emergency, the patient might not trust that a first responder is, in fact, a first responder with good intentions. The lack of trust can complicate efforts to administer care to the patient.

### BRIEF DESCRIPTION OF THE DRAWINGS

Reference will now be made to the accompanying drawings, which are not necessarily drawn to scale, and wherein:

FIG. 1 is a block diagram illustrating an exemplary, non-limiting embodiment of a communications network in accordance with various aspects described herein.

FIG. 2A is a block diagram illustrating an example, non-limiting embodiment of a system functioning within the communication network of FIG. 1 in accordance with various aspects described herein.

FIG. 2B depicts an illustrative embodiment of a method in accordance with various aspects described herein.

FIG. 3 is a block diagram illustrating an example, non-limiting embodiment of a virtualized communication network in accordance with various aspects described herein.

FIG. 4 is a block diagram of an example, non-limiting embodiment of a computing environment in accordance with various aspects described herein.

FIG. 5 is a block diagram of an example, non-limiting embodiment of a mobile network platform in accordance with various aspects described herein.

FIG. 6 is a block diagram of an example, non-limiting embodiment of a communication device in accordance with various aspects described herein.

### DETAILED DESCRIPTION

The subject disclosure describes, among other things, illustrative embodiments for granting and providing conditional access to data on an as-needed basis via the use of permissions or keys. Other embodiments are described in the subject disclosure.

One or more aspects of the subject disclosure include, in whole or in part, obtaining a first request for medical attention to be administered to a first user, wherein the first user is in possession of a first communication device that stores first data, transmitting an identifier associated with the first request, wherein the identifier is received by a second communication device associated with a second user, obtaining a second request from the second communication device, the second request including the identifier, based on the obtaining of the second request, generating a first key based on second data associated with the second user, and transmitting the first key to the second communication device such that the second communication device obtains a first portion of the first data from the first communication device by transmitting the first key to the first communication device.

One or more aspects of the subject disclosure include, in whole or in part, transmitting an identifier associated with a first request for medical attention to be administered to a first user, wherein the first user is associated with a first user equipment that stores first data pertaining to a medical history of the first user, and wherein the identifier is received by a second user equipment associated with a second user and a third user equipment associated with a third user, obtaining a second request from the second user equipment, the second request including the identifier, based on the obtaining of the second request, generating a first key based on second data associated with the second user, wherein the first key grants the second user equipment access to a first portion of the first data, obtaining a third request from the third user equipment, the third request including the identifier, based on the obtaining of the third request, generating a second key based on third data associated with the third user, wherein the second key grants the third user equipment access to a second portion of the first data, the second portion of the first data being at least partially different from the first portion of the first data, transmitting the first key to the second user equipment, and transmitting the second key to the third user equipment.

One or more aspects of the subject disclosure include, in whole or in part, transmitting, by a processing system including a processor, a request for attention, wherein the processing system stores first data pertaining to a first user, receiving, by the processing system and based on the transmitting of the request for attention, a key from a second processing system including a second processor, based on the receiving of the key, identifying, by the processing system, a first portion of the first data to be transmitted to the second processing system, wherein the first portion of the first data is less than an entirety of the first data, and based on the identifying of the first portion of the first data, transmitting, by the processing system, the first portion of the first data to the second processing system.

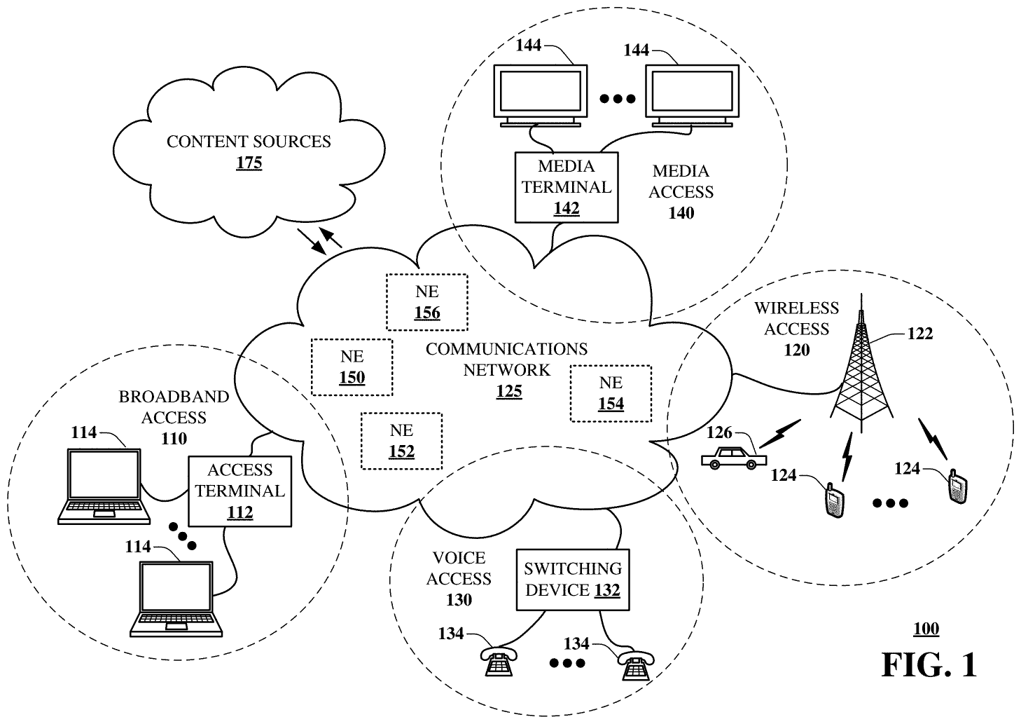

Referring now to FIG. 1, a block diagram is shown illustrating an example, non-limiting embodiment of a system 100 in accordance with various aspects described herein. For example, system 100 can facilitate in whole or in part obtaining a first request for medical attention to be administered to a first user, wherein the first user is in possession of a first communication device that stores first data, transmitting an identifier associated with the first request, wherein the identifier is received by a second communication device associated with a second user, obtaining a second request from the second communication device, the second request including the identifier, based on the obtaining of the second request, generating a first key based on second data associated with the second user, and transmitting the first key to the second communication device such that the second communication device obtains a first portion of the first data from the first communication device by transmitting the first key to the first communication device. System 100 can facilitate in whole or in part transmitting an identifier associated with a first request for medical attention to be administered to a first user, wherein the first user is associated with a first user equipment that stores first data pertaining to a medical history of the first user, and wherein the identifier is received by a second user equipment associated with a second user and a third user equipment associated with a third user, obtaining a second request from the second user equipment, the second request including the identifier, based on the obtaining of the second request, generating a first key based on second data associated with the second user, wherein the first key grants the second user equipment access to a first portion of the first data, obtaining a third request from the third user equipment, the third request including the identifier, based on the obtaining of the third request, generating a second key based on third data associated with the third user, wherein the second key grants the third user equipment access to a second portion of the first data, the second portion of the first data being at least partially different from the first portion of the first data, transmitting the first key to the second user equipment, and transmitting the second key to the third user equipment. System 100 can facilitate in whole or in part transmitting, by a processing system including a processor, a request for attention, wherein the processing system stores first data pertaining to a first user, receiving, by the processing system and based on the transmitting of the request for attention, a key from a second processing system including a second processor, based on the receiving of the key, identifying, by the processing system, a first portion of the first data to be transmitted to the second processing system, wherein the first portion of the first data is less than an entirety of the first data, and based on the identifying of the first portion of the first data, transmitting, by the processing system, the first portion of the first data to the second processing system.

In particular, in FIG. 1 a communications network 125 is presented for providing broadband access 110 to a plurality of data terminals 114 via access terminal 112, wireless access 120 to a plurality of mobile devices 124 and vehicle 126 via base station or access point 122, voice access 130 to a plurality of telephony devices 134, via switching device 132 and/or media access 140 to a plurality of audio/video display devices 144 via media terminal 142. In addition, communication network 125 is coupled to one or more content sources 175 of audio, video, graphics, text and/or other media. While broadband access 110, wireless access 120, voice access 130 and media access 140 are shown separately, one or more of these forms of access can be combined to provide multiple access services to a single client device (e.g., mobile devices 124 can receive media content via media terminal 142, data terminal 114 can be provided voice access via switching device 132, and so on).

The communications network 125 includes a plurality of network elements (NE) 150, 152, 154, 156, etc. for facilitating the broadband access 110, wireless access 120, voice access 130, media access 140 and/or the distribution of content from content sources 175. The communications network 125 can include a circuit switched or packet switched network, a voice over Internet protocol (VoIP) network, Internet protocol (IP) network, a cable network, a passive or active optical network, a 4G, 5G, or higher generation wireless access network, WIMAX network, UltraWideband network, personal area network or other wireless access network, a broadcast satellite network and/or other communications network.

In various embodiments, the access terminal 112 can include a digital subscriber line access multiplexer (DSLAM), cable modem termination system (CMTS), optical line terminal (OLT) and/or other access terminal. The data terminals 114 can include personal computers, laptop computers, netbook computers, tablets or other computing devices along with digital subscriber line (DSL) modems, data over coax service interface specification (DOCSIS) modems or other cable modems, a wireless modem such as a 4G, 5G, or higher generation modem, an optical modem and/or other access devices.

In various embodiments, the base station or access point 122 can include a 4G, 5G, or higher generation base station, an access point that operates via an 802.11 standard such as 802.11n, 802.11ac or other wireless access terminal. The mobile devices 124 can include mobile phones, e-readers, tablets, phablets, wireless modems, and/or other mobile computing devices.

In various embodiments, the switching device 132 can include a private branch exchange or central office switch, a media services gateway, VoIP gateway or other gateway device and/or other switching device. The telephony devices 134 can include traditional telephones (with or without a terminal adapter), VoIP telephones and/or other telephony devices.

In various embodiments, the media terminal 142 can include a cable head-end or other TV head-end, a satellite receiver, gateway or other media terminal 142. The display devices 144 can include televisions with or without a set top box, personal computers and/or other display devices.

In various embodiments, the content sources 175 include broadcast television and radio sources, video on demand platforms and streaming video and audio services platforms, one or more content data networks, data servers, web servers and other content servers, and/or other sources of media.

In various embodiments, the communications network 125 can include wired, optical and/or wireless links and the network elements 150, 152, 154, 156, etc. can include service switching points, signal transfer points, service control points, network gateways, media distribution hubs, servers, firewalls, routers, edge devices, switches and other network nodes for routing and controlling communications traffic over wired, optical and wireless links as part of the Internet and other public networks as well as one or more private networks, for managing subscriber access, for billing and network management and for supporting other network functions.

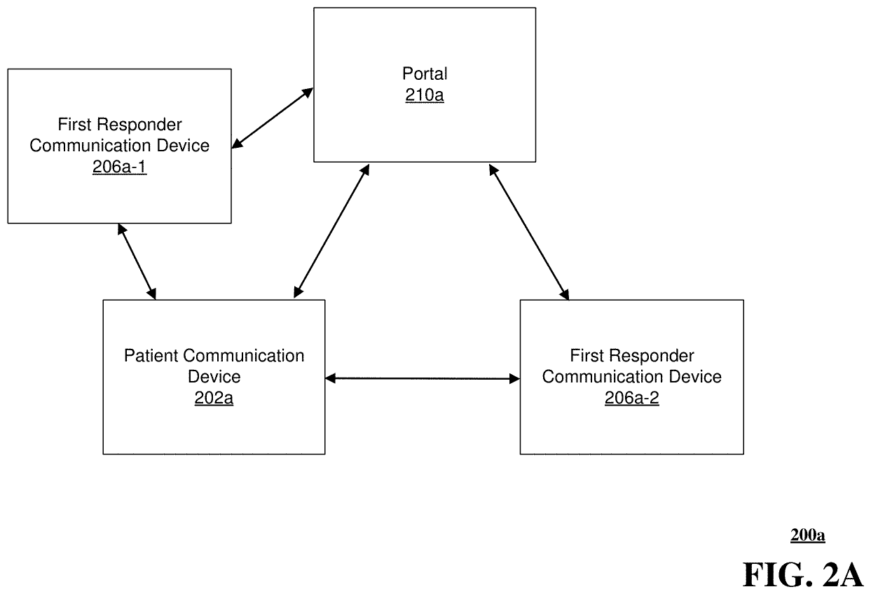

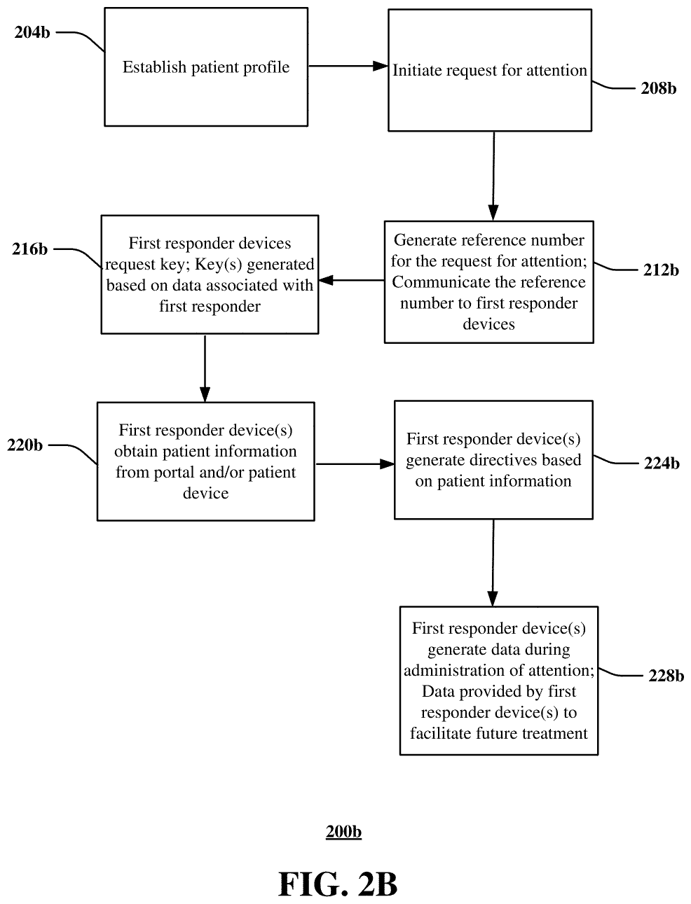

FIG. 2A is a block diagram illustrating an example, non-limiting embodiment of a system 200a in accordance with various aspects described herein. The system 200a may function within, or may be operatively overlaid upon, the communication network 100 of FIG. 1. The system 200a is described below in relation to a scenario pertaining to an administration of medical care to a patient by one or more first responders during an emergency. One skilled in the art will appreciate that aspects of the system 200a may be implemented in relation to other scenarios, inclusive of a first scenario directed to routine medical treatment and other scenarios that do not even pertain to medical treatment.

It must be borne in mind that the scenarios/situations described herein are purely and solely of a theoretical/hypothetical nature. The scenarios do not necessarily include/encompass factual conditions or circumstances that may warrant a plan of treatment or action that differs from what is stated herein. Nothing stated herein constitutes medical advice or medical recommendations, in a general sense or in relation to specific circumstances or conditions. Readers/Reviewers of this disclosure are encouraged to consult with trained professionals (e.g., licensed medical professionals) as part of seeking out, or obtaining, treatment or advice in connection with emergency and non-emergency situations. That said, nothing stated herein represents an endorsement or approval of any statements or opinions that may be expressed by such professionals.

Referring back to FIG. 2A, the system 200a may include a patient communication device 202a, first responder communication devices 206a-1 and 206a-2, and a portal 210a. The entities 202a, 206a-1, 206a-2, and 210a may be representative of any number or type of communication device(s); the nomenclature used in relation to such entities is exemplary.

In some embodiments, the patient communication device 202a may include one or more wearable devices, such as for example a wristwatch, a biological sensor, etc. The patient communication device 202a may include a portable/mobile electronic device, such as a smartphone. The first responder communication devices 206a-1 and 206a-2 may include one or more mobile electronic devices.

The portal 210a may store first data indicative of patient records, treatment plans, and the like, for one or more users/patients. The first data may be developed, in whole or in part, in consultation with one or more professionals, inclusive of one or more medical professionals. The portal 210a may store second data indicative of credentials associated with one or more users/first responders, such as for example a 1^{st} first responder associated with the first responder communication device 206a-1 and a 2^{nd} first responder associated with the first responder communication device 206a-2. The credentials associated with the first responder(s) may include an identification of: licenses, accreditations, or certifications/certificates awarded, skills/skill sets or experience possessed, courses/classes/seminars/training taken or completed, etc. The portal 210a may store data (e.g., the first data and/or the second data) in accordance with one or more identifiers, which is to say that separate profiles may be maintained in the portal 210a to distinguish one user from another user (e.g., to distinguish a first user/patient associated with the patient communication device 202a from a second user/patient (not shown in FIG. 2A); to distinguish the 1^{st} first responder from the 2^{nd} first responder, etc.).

The patient communication device 202a may be communicatively coupled to the portal 210a to facilitate an exchange of medical information or data pertinent to the user/patient associated with the patient communication device 202a. For example, data/information pertaining to user/patient may be uploaded from the patient communication device 202a (or another communication device) to the portal 210a or downloaded from the portal 210a to the patient communication device 202a (or another communication device). Similarly, each of the first responder communication devices 206a-1 and 206a-2 may be communicatively coupled to the portal 210a to facilitate an exchange of data pertinent to first responders associated with each of the devices 206a-1 and 206a-2. For reasons that will become clearer below, one or both of the first responder communication devices 206a-1 and 206a-2 may be communicatively coupled to the patient communication device 202a for a given time duration, potentially over one or more local networks and/or via one or more local communication links.

In a particular, exemplary scenario it may be assumed that a user/patient associated with the patient communication device 202a has incurred an emergency medical situation. For example, the patient may have previously been diagnosed as being diabetic and she may have slipped into diabetic shock. Further, it may be assumed that the patient's heart has ceased beating or is beating at a rate that is less than average/normal, and that the patient would benefit from an administration of cardiopulmonary resuscitation (CPR). For purposes of further illustrating and describing this scenario, reference may be made to FIG. 2B, which depicts an illustrative embodiment of a method 200b in accordance with various aspects described herein. Various blocks or operations of the method 200b are described in further detail below. In brief, block 204b provides for an establishment of a medical record/profile for the patient in the portal 210a. The profile may be populated with information pertaining to the patient, such as an identification that the patient has been diagnosed as being diabetic. Other information pertinent to the patient, inclusive of other information associated with the patient's medical history, may be captured/included in the profile for the patient. To demonstrate, the information included in the profile for the patient may include other conditions/afflictions that the patient suffers from (e.g., high cholesterol levels, elevated blood pressure, etc.), allergies to medications, etc.

The patient's need for medical treatment in this particular scenario may be detected/triggered by the patient communication device 202a. For example, a sensor associated with the patient communication device 202a may detect a rapid descent of the patient (e.g., a descent at a rate greater than a threshold) that may indicate that the patient has fallen/collapsed. The detection of this rapid descent may initiate/generate a request for treatment/assistance that may be transmitted by the patient communication device 202a. The request may be received by, e.g., the portal 210a (or another device/entity).

The output of a sensor of the patient communication device 202a driving the generation of the request is one example of how the request may be triggered/initiated. In another instance/scenario, the patient may indicate a need or desire for assistance by manually initiating the request on her patient communication device 202a. In yet another instance/scenario, a passerby may witness the patient falling and may initiate a request for assistance on his own device on behalf of the patient. In general, a need or desire for medical attention (or other types of attention, inclusive of emergency attention) may be initiated/triggered as part of block 208b using any number or types of technique(s).

In some embodiments, and to the extent that information pertaining to the scenario/situation is known, the request of block 208b may include an identification of the types of conditions surrounding/associated with the scenario. For example, the request may include an identification that the patient has fallen (as detected by the sensor associated with the patient communication device 202a, for example). The request of block 208b may include an identification of a location of the patient. Techniques for locating a user or device, such as geo-locating applications, GPS applications, and the like, are known to one of skill in the art; as such, a complete description of such techniques is omitted herein for the sake of brevity.

Based on the request for attention generated as part of block 208b, the portal 210a may generate a reference number (or other identifier) that is associated with the request for attention. The reference number may be used to distinguish the scenario involving the patient with other scenarios, inclusive of other scenarios involving that same user/patient and other users/patients.

As part of block 212b, the reference number assigned/allocated to the particular scenario in question may be communicated to one or more first responders (or, analogously, one or more communication devices associated with the first responders, such as the devices 206a-1 and 206a-2 of FIG. 2A). In some embodiments, a first responder may have an option to accept or decline an assignment of rendering assistance to the patient, such as for example when the first responder is not scheduled to work (e.g., when the first is not “on the clock”). In some embodiments, the communication of the reference number to first responders may be limited to those first responders that are proximal to the patient (e.g., may be limited to those first responders that are within a threshold distance of the patient, or may be limited to one or more first responders that are closest to the location of the patient). In this way, first responders will not be bothered with requests for assistance that are likely to be handled by other first responders.

In block 216b, and for those first responders that received the identifier/reference number of block 212b (and chose to accept the assignment of rendering assistance, where applicable), the first responder device(s) may request a key. As described in further detail below, a key may be used by a first responder device to unlock information associated with the patient. A key may be generated for a particular first responder as part of block 216b. For example, the key that is generated for a particular first responder may be based on the second data stored in the portal 210a for that first responder as described above. Stated differently, as part of block 216b a first key may be generated for a 1^{st} first responder and a second key may be generated for a 2^{nd} first responder; the second key may the same as, or at least partially different from, the first key.

Block 216b may include providing/transmitting keys to respective communication devices associated with first responders. To demonstrate, and continuing the above example, block 216b may include transmitting the first key to the first responder communication device 206a-1 and transmitting the second key to the first responder communication device 206a-2.

In block 220b, each of the first responder communication devices that obtained keys (as part of block 216b) may initiate a respective communication that may be detected by, e.g., the portal 210a and/or the patient communication device 202a. The initiated communication(s) of block 220b may include the key associated with the respective first responder communication device. The key may be used to unlock/obtain a set of patient data/information identified by the key. Thus, in block 220b the first key transmitted by the first responder communication device 206a-1 may result in the first responder communication device 206a-1 obtaining a first set of data/information that is pertinent to the patient. In block 220b the second key transmitted by the first responder communication device 206a-2 may result in the first responder communication device 206a-2 obtaining a second set of data/information that is pertinent to the patient. If the first key and the second key are the same, then the first set of data/information and the second set of data/information may be the same; otherwise, if the first key and the second key are at least partially different from one another, the first set of data/information and the second set of data/information may be at least partially different from one another.

In block 224b, the first responder communication device(s) that obtained the set(s) of patient data/information as part of block 220b may analyze that patient data/information, potentially in combination with other data/information that is pertinent to the respective first responder (such as credentials associated with the first responder). Based on the analysis, the first responder communication device(s) may generate and present respective instructions/directives/commands for the first responder as to what to do in the scenario/situation.

Continuing the above example pertaining to two first responders, and assuming that each first responder (communication device) obtained a different key as part of block 216b (and thus, obtained different sets of data/information as part of block 220b), and further assuming that the first responder associated with the first responder communication device 206a-1 is trained in CPR, a first directive generated by the first responder communication device 206a-1 in block 224b may instruct/command the first responder to perform a task, such as administering CPR to the patient. Assuming that the first responder associated with the first responder communication device 206a-2 is not trained in administering CPR (or assuming that the first responder associated with the first responder communication device 206a-1 is better equipped/trained to administer CPR relative to the first responder associated with the first responder communication device 206a-2), a second directive generated by the first responder communication device 206a-2 in block 224b may instruct/command the first responder associated with the first responder communication device 206a-2 to undertake other tasks, such as preparing medications to be administered to the patient, preparing an oxygen tank/apparatus to be administered to the patient, and calling for backup support.

In block 228b, the first responder communication devices may generate data during the administration/performance of tasks directed to the attention that was requested/solicited (e.g., the medical attention that was requested). The data generated in block 228b may be shared with, e.g., the patient communication device 202a and/or the portal 210a in order to ensure that the patient's records are kept current/up-to-date, which may be useful in terms of facilitating future treatment, plans, etc., in the care of the patient. For example, the data of block 228b may be analyzed by the patient's doctor to chart-out/plan future treatment.

In some embodiments, the keys generated in block 216b may include restrictions/limitations in terms of what can be done with a patient's data/information as obtained as part of block 220b. For example, in some instances a key may prohibit the respective first responder communication device from: (1) storing the patient data/information in a memory device of the first responder communication device, (2) transmitting the patient data/information, (3) editing the patient data/information, etc., or any combination thereof. In some embodiments, at least some of the restrictions/limitations may be included/incorporated as part of one or more applications executed by a first responder communication device, which is to say that some or all of the restrictions/limitations might not be present in the key(s).

In some embodiments, a key may expire. For example, a key may expire after a certain timeout/time period following its generation (subject to a potential renewal/reinstatement procedure). A key may expire based on an occurrence of one or more events or conditions, such as for example a patient or portal (or associated communication device) revoking the key, etc. Revocation of a key may result in patient data/information that is accessible by a first responder communication device no longer being accessible by that first responder communication device, which is to say that patient data/information might only be accessible by the first responder communication device for the duration that it is needed.

While for purposes of simplicity of explanation, the respective processes are shown and described as a series of blocks in FIG. 2B, it is to be understood and appreciated that the claimed subject matter is not limited by the order of the blocks, as some blocks may occur in different orders and/or concurrently with other blocks from what is depicted and described herein. Moreover, not all illustrated blocks may be required to implement the methods described herein.

As the foregoing demonstrates, aspects of this disclosure may facilitate an exchange or provisioning of data/information associated with a user (e.g., a patient) on an as-needed basis. For example, and in relation to the scenario described above of a diabetic patient, data/information associated with the patient was shared with first responders (or associated first responder communication devices) only to the extent that was necessary for administering care. In this respect, it is noted that the first responders in the above scenario might not have been aware of certain data/information contained in a patient's records, such as the patient's name, birthday, residential address, etc. Still further, and as described above, access to patient data/information may be restricted/limited in terms of time/duration to reduce (e.g., minimize) the amount of exposure of such data/information.

As set forth above, principles of security and privacy may be enhanced/maintained in respect of user/patient data/information, while still ensuring that first responders have access to such data/information to the extent that such data/information is pertinent to an administration of care. Taken in total, and as the foregoing demonstrates, aspects of this disclosure represent substantial improvements in terms of the administration of care afforded to users/patients by presenting data/information to first responders that is relevant to the tasks that are performed by the first responders. Stated differently, in conventional settings first responders frequently need to sort through and filter-out irrelevant data in order to identify relevant data that is pertinent to the tasks that the first responders are to perform. Aspects of this disclosure alleviate the need for first responders to engage in such laborious and time-consuming activities and reduce the likelihood of human error having a detrimental impact on the administration of care.

As the foregoing examples demonstrate, aspects of this disclosure may be included/incorporated as part of one or more practical applications. For example, and as described above, data/information associated with a user/patient and/or a user/first responder may be utilized to identify tasks that are to be performed by the first responder in conjunction with administering care to the patient. One skilled in the art will appreciate that aspects of this disclosure may be included/incorporated as part of one or more other applications. For example, aspects of this disclosure may be utilized as part of one or more network operations to create a handshake/agreement between devices such that a first device can know what other device(s) the first device is interfacing with and facilitate a secure (e.g., encrypted) exchange of data/information to realize particular results/objectives.

In some embodiments, one or more identifiers may be used to identify a device where data should be sent to. In this respect, an identifier (such as an address, a make/model/serial number, etc.) associated with a device may be used to distinguish the device from other devices. Use of such an identifier may help to ensure that the data is only sent to (or acted upon) by an appropriate device under the circumstances/conditions that are present.

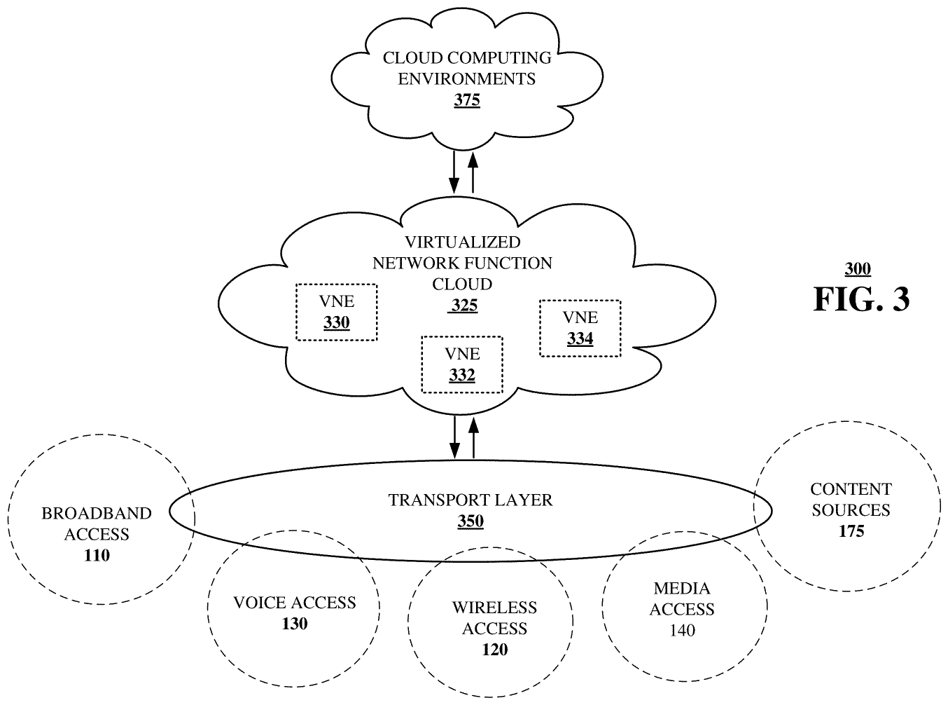

Referring now to FIG. 3, a block diagram 300 is shown illustrating an example, non-limiting embodiment of a virtualized communication network in accordance with various aspects described herein. In particular a virtualized communication network is presented that can be used to implement some or all of the subsystems and functions of system 100, the subsystems and functions of system 200a, and method 200b presented in FIGS. 1, 2A, and 2B. For example, virtualized communication network 300 can facilitate in whole or in part obtaining a first request for medical attention to be administered to a first user, wherein the first user is in possession of a first communication device that stores first data, transmitting an identifier associated with the first request, wherein the identifier is received by a second communication device associated with a second user, obtaining a second request from the second communication device, the second request including the identifier, based on the obtaining of the second request, generating a first key based on second data associated with the second user, and transmitting the first key to the second communication device such that the second communication device obtains a first portion of the first data from the first communication device by transmitting the first key to the first communication device. Virtualized communication network 300 can facilitate in whole or in part transmitting an identifier associated with a first request for medical attention to be administered to a first user, wherein the first user is associated with a first user equipment that stores first data pertaining to a medical history of the first user, and wherein the identifier is received by a second user equipment associated with a second user and a third user equipment associated with a third user, obtaining a second request from the second user equipment, the second request including the identifier, based on the obtaining of the second request, generating a first key based on second data associated with the second user, wherein the first key grants the second user equipment access to a first portion of the first data, obtaining a third request from the third user equipment, the third request including the identifier, based on the obtaining of the third request, generating a second key based on third data associated with the third user, wherein the second key grants the third user equipment access to a second portion of the first data, the second portion of the first data being at least partially different from the first portion of the first data, transmitting the first key to the second user equipment, and transmitting the second key to the third user equipment. Virtualized communication network 300 can facilitate in whole or in part transmitting, by a processing system including a processor, a request for attention, wherein the processing system stores first data pertaining to a first user, receiving, by the processing system and based on the transmitting of the request for attention, a key from a second processing system including a second processor, based on the receiving of the key, identifying, by the processing system, a first portion of the first data to be transmitted to the second processing system, wherein the first portion of the first data is less than an entirety of the first data, and based on the identifying of the first portion of the first data, transmitting, by the processing system, the first portion of the first data to the second processing system.

In particular, a cloud networking architecture is shown that leverages cloud technologies and supports rapid innovation and scalability via a transport layer 350, a virtualized network function cloud 325 and/or one or more cloud computing environments 375. In various embodiments, this cloud networking architecture is an open architecture that leverages application programming interfaces (APIs); reduces complexity from services and operations; supports more nimble business models; and rapidly and seamlessly scales to meet evolving customer requirements including traffic growth, diversity of traffic types, and diversity of performance and reliability expectations.

In contrast to traditional network elements—which are typically integrated to perform a single function, the virtualized communication network employs virtual network elements (VNEs) 330, 332, 334, etc. that perform some or all of the functions of network elements 150, 152, 154, 156, etc. For example, the network architecture can provide a substrate of networking capability, often called Network Function Virtualization Infrastructure (NFVI) or simply infrastructure that is capable of being directed with software and Software Defined Networking (SDN) protocols to perform a broad variety of network functions and services. This infrastructure can include several types of substrates. The most typical type of substrate being servers that support Network Function Virtualization (NFV), followed by packet forwarding capabilities based on generic computing resources, with specialized network technologies brought to bear when general purpose processors or general purpose integrated circuit devices offered by merchants (referred to herein as merchant silicon) are not appropriate. In this case, communication services can be implemented as cloud-centric workloads.

As an example, a traditional network element 150 (shown in FIG. 1), such as an edge router can be implemented via a VNE 330 composed of NFV software modules, merchant silicon, and associated controllers. The software can be written so that increasing workload consumes incremental resources from a common resource pool, and moreover so that it's elastic: so the resources are only consumed when needed. In a similar fashion, other network elements such as other routers, switches, edge caches, and middle-boxes are instantiated from the common resource pool. Such sharing of infrastructure across a broad set of uses makes planning and growing infrastructure easier to manage.

In an embodiment, the transport layer 350 includes fiber, cable, wired and/or wireless transport elements, network elements and interfaces to provide broadband access 110, wireless access 120, voice access 130, media access 140 and/or access to content sources 175 for distribution of content to any or all of the access technologies. In particular, in some cases a network element needs to be positioned at a specific place, and this allows for less sharing of common infrastructure. Other times, the network elements have specific physical layer adapters that cannot be abstracted or virtualized, and might require special DSP code and analog front-ends (AFEs) that do not lend themselves to implementation as VNEs 330, 332 or 334. These network elements can be included in transport layer 350.

The virtualized network function cloud 325 interfaces with the transport layer 350 to provide the VNEs 330, 332, 334, etc. to provide specific NFVs. In particular, the virtualized network function cloud 325 leverages cloud operations, applications, and architectures to support networking workloads. The virtualized network elements 330, 332 and 334 can employ network function software that provides either a one-for-one mapping of traditional network element function or alternately some combination of network functions designed for cloud computing. For example, VNEs 330, 332 and 334 can include route reflectors, domain name system (DNS) servers, and dynamic host configuration protocol (DHCP) servers, system architecture evolution (SAE) and/or mobility management entity (MME) gateways, broadband network gateways, IP edge routers for IP-VPN, Ethernet and other services, load balancers, distributers and other network elements. Because these elements don't typically need to forward large amounts of traffic, their workload can be distributed across a number of servers—each of which adds a portion of the capability, and overall which creates an elastic function with higher availability than its former monolithic version. These virtual network elements 330, 332, 334, etc. can be instantiated and managed using an orchestration approach similar to those used in cloud compute services.

The cloud computing environments 375 can interface with the virtualized network function cloud 325 via APIs that expose functional capabilities of the VNEs 330, 332, 334, etc. to provide the flexible and expanded capabilities to the virtualized network function cloud 325. In particular, network workloads may have applications distributed across the virtualized network function cloud 325 and cloud computing environment 375 and in the commercial cloud, or might simply orchestrate workloads supported entirely in NFV infrastructure from these third party locations.

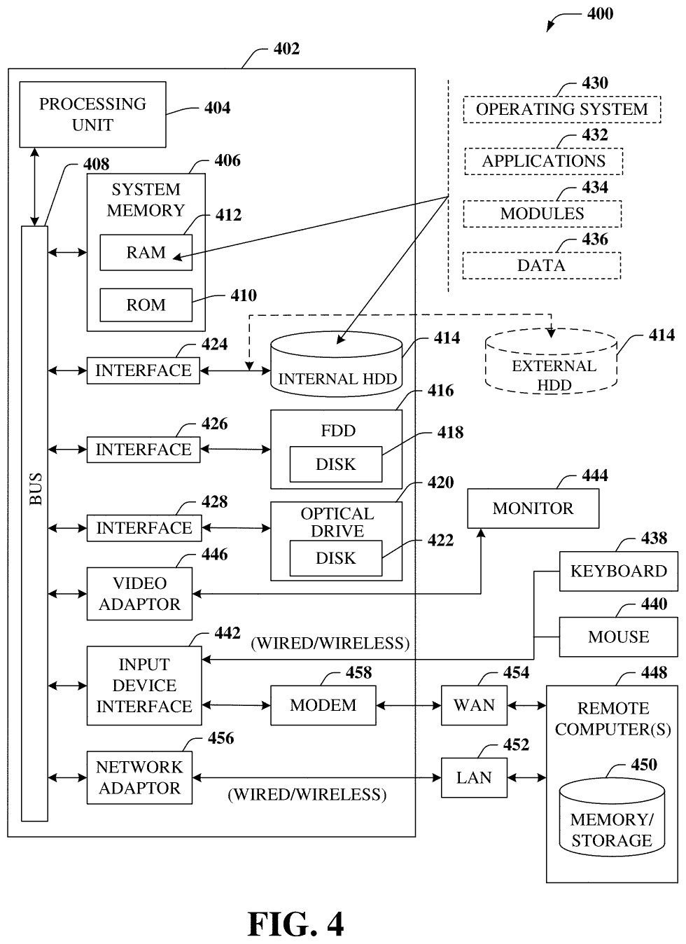

Turning now to FIG. 4, there is illustrated a block diagram of a computing environment in accordance with various aspects described herein. In order to provide additional context for various embodiments of the embodiments described herein, FIG. 4 and the following discussion are intended to provide a brief, general description of a suitable computing environment 400 in which the various embodiments of the subject disclosure can be implemented. In particular, computing environment 400 can be used in the implementation of network elements 150, 152, 154, 156, access terminal 112, base station or access point 122, switching device 132, media terminal 142, and/or VNEs 330, 332, 334, etc. Each of these devices can be implemented via computer-executable instructions that can run on one or more computers, and/or in combination with other program modules and/or as a combination of hardware and software. For example, computing environment 400 can facilitate in whole or in part obtaining a first request for medical attention to be administered to a first user, wherein the first user is in possession of a first communication device that stores first data, transmitting an identifier associated with the first request, wherein the identifier is received by a second communication device associated with a second user, obtaining a second request from the second communication device, the second request including the identifier, based on the obtaining of the second request, generating a first key based on second data associated with the second user, and transmitting the first key to the second communication device such that the second communication device obtains a first portion of the first data from the first communication device by transmitting the first key to the first communication device. Computing environment 400 can facilitate in whole or in part transmitting an identifier associated with a first request for medical attention to be administered to a first user, wherein the first user is associated with a first user equipment that stores first data pertaining to a medical history of the first user, and wherein the identifier is received by a second user equipment associated with a second user and a third user equipment associated with a third user, obtaining a second request from the second user equipment, the second request including the identifier, based on the obtaining of the second request, generating a first key based on second data associated with the second user, wherein the first key grants the second user equipment access to a first portion of the first data, obtaining a third request from the third user equipment, the third request including the identifier, based on the obtaining of the third request, generating a second key based on third data associated with the third user, wherein the second key grants the third user equipment access to a second portion of the first data, the second portion of the first data being at least partially different from the first portion of the first data, transmitting the first key to the second user equipment, and transmitting the second key to the third user equipment. Computing environment 400 can facilitate in whole or in part transmitting, by a processing system including a processor, a request for attention, wherein the processing system stores first data pertaining to a first user, receiving, by the processing system and based on the transmitting of the request for attention, a key from a second processing system including a second processor, based on the receiving of the key, identifying, by the processing system, a first portion of the first data to be transmitted to the second processing system, wherein the first portion of the first data is less than an entirety of the first data, and based on the identifying of the first portion of the first data, transmitting, by the processing system, the first portion of the first data to the second processing system.

Generally, program modules comprise routines, programs, components, data structures, etc., that perform particular tasks or implement particular abstract data types. Moreover, those skilled in the art will appreciate that the methods can be practiced with other computer system configurations, comprising single-processor or multiprocessor computer systems, minicomputers, mainframe computers, as well as personal computers, hand-held computing devices, microprocessor-based or programmable consumer electronics, and the like, each of which can be operatively coupled to one or more associated devices.

As used herein, a processing circuit includes one or more processors as well as other application specific circuits such as an application specific integrated circuit, digital logic circuit, state machine, programmable gate array or other circuit that processes input signals or data and that produces output signals or data in response thereto. It should be noted that while any functions and features described herein in association with the operation of a processor could likewise be performed by a processing circuit.

The illustrated embodiments of the embodiments herein can be also practiced in distributed computing environments where certain tasks are performed by remote processing devices that are linked through a communications network. In a distributed computing environment, program modules can be located in both local and remote memory storage devices.

Computing devices typically comprise a variety of media, which can comprise computer-readable storage media and/or communications media, which two terms are used herein differently from one another as follows. Computer-readable storage media can be any available storage media that can be accessed by the computer and comprises both volatile and nonvolatile media, removable and non-removable media. By way of example, and not limitation, computer-readable storage media can be implemented in connection with any method or technology for storage of information such as computer-readable instructions, program modules, structured data or unstructured data.

Computer-readable storage media can comprise, but are not limited to, random access memory (RAM), read only memory (ROM), electrically erasable programmable read only memory (EEPROM),flash memory or other memory technology, compact disk read only memory (CD-ROM), digital versatile disk (DVD) or other optical disk storage, magnetic cassettes, magnetic tape, magnetic disk storage or other magnetic storage devices or other tangible and/or non-transitory media which can be used to store desired information. In this regard, the terms “tangible” or “non-transitory” herein as applied to storage, memory or computer-readable media, are to be understood to exclude only propagating transitory signals per se as modifiers and do not relinquish rights to all standard storage, memory or computer-readable media that are not only propagating transitory signals per se.

Computer-readable storage media can be accessed by one or more local or remote computing devices, e.g., via access requests, queries or other data retrieval protocols, for a variety of operations with respect to the information stored by the medium.

Communications media typically embody computer-readable instructions, data structures, program modules or other structured or unstructured data in a data signal such as a modulated data signal, e.g., a carrier wave or other transport mechanism, and comprises any information delivery or transport media. The term “modulated data signal” or signals refers to a signal that has one or more of its characteristics set or changed in such a manner as to encode information in one or more signals. By way of example, and not limitation, communication media comprise wired media, such as a wired network or direct-wired connection, and wireless media such as acoustic, RF, infrared and other wireless media.

With reference again to FIG. 4, the example environment can comprise a computer 402, the computer 402 comprising a processing unit 404, a system memory 406 and a system bus 408. The system bus 408 couples system components including, but not limited to, the system memory 406 to the processing unit 404. The processing unit 404 can be any of various commercially available processors. Dual microprocessors and other multiprocessor architectures can also be employed as the processing unit 404.

The system bus 408 can be any of several types of bus structure that can further interconnect to a memory bus (with or without a memory controller), a peripheral bus, and a local bus using any of a variety of commercially available bus architectures. The system memory 406 comprises ROM 410 and RAM 412. A basic input/output system (BIOS) can be stored in a non-volatile memory such as ROM, erasable programmable read only memory (EPROM), EEPROM, which BIOS contains the basic routines that help to transfer information between elements within the computer 402, such as during startup. The RAM 412 can also comprise a high-speed RAM such as static RAM for caching data.

The computer 402 further comprises an internal hard disk drive (HDD) 414 (e.g., EIDE, SATA), which internal HDD 414 can also be configured for external use in a suitable chassis (not shown), a magnetic floppy disk drive (FDD) 416, (e.g., to read from or write to a removable diskette 418) and an optical disk drive 420, (e.g., reading a CD-ROM disk 422 or, to read from or write to other high capacity optical media such as the DVD). The HDD 414, magnetic FDD 416 and optical disk drive 420 can be connected to the system bus 408 by a hard disk drive interface 424, a magnetic disk drive interface 426 and an optical drive interface 428, respectively. The hard disk drive interface 424 for external drive implementations comprises at least one or both of Universal Serial Bus (USB) and Institute of Electrical and Electronics Engineers (IEEE) 1394 interface technologies. Other external drive connection technologies are within contemplation of the embodiments described herein.

The drives and their associated computer-readable storage media provide nonvolatile storage of data, data structures, computer-executable instructions, and so forth. For the computer 402, the drives and storage media accommodate the storage of any data in a suitable digital format. Although the description of computer-readable storage media above refers to a hard disk drive (HDD), a removable magnetic diskette, and a removable optical media such as a CD or DVD, it should be appreciated by those skilled in the art that other types of storage media which are readable by a computer, such as zip drives, magnetic cassettes, flash memory cards, cartridges, and the like, can also be used in the example operating environment, and further, that any such storage media can contain computer-executable instructions for performing the methods described herein.

A number of program modules can be stored in the drives and RAM 412, comprising an operating system 430, one or more application programs 432, other program modules 434 and program data 436. All or portions of the operating system, applications, modules, and/or data can also be cached in the RAM 412. The systems and methods described herein can be implemented utilizing various commercially available operating systems or combinations of operating systems.

A user can enter commands and information into the computer 402 through one or more wired/wireless input devices, e.g., a keyboard 438 and a pointing device, such as a mouse 440. Other input devices (not shown) can comprise a microphone, an infrared (IR) remote control, a joystick, a game pad, a stylus pen, touch screen or the like. These and other input devices are often connected to the processing unit 404 through an input device interface 442 that can be coupled to the system bus 408, but can be connected by other interfaces, such as a parallel port, an IEEE 1394 serial port, a game port, a universal serial bus (USB) port, an IR interface, etc.

A monitor 444 or other type of display device can be also connected to the system bus 408 via an interface, such as a video adapter 446. It will also be appreciated that in alternative embodiments, a monitor 444 can also be any display device (e.g., another computer having a display, a smart phone, a tablet computer, etc.) for receiving display information associated with computer 402 via any communication means, including via the Internet and cloud-based networks. In addition to the monitor 444, a computer typically comprises other peripheral output devices (not shown), such as speakers, printers, etc.

The computer 402 can operate in a networked environment using logical connections via wired and/or wireless communications to one or more remote computers, such as a remote computer(s) 448. The remote computer(s) 448 can be a workstation, a server computer, a router, a personal computer, portable computer, microprocessor-based entertainment appliance, a peer device or other common network node, and typically comprises many or all of the elements described relative to the computer 402, although, for purposes of brevity, only a remote memory/storage device 450 is illustrated. The logical connections depicted comprise wired/wireless connectivity to a local area network (LAN) 452 and/or larger networks, e.g., a wide area network (WAN) 454. Such LAN and WAN networking environments are commonplace in offices and companies, and facilitate enterprise-wide computer networks, such as intranets, all of which can connect to a global communications network, e.g., the Internet.

When used in a LAN networking environment, the computer 402 can be connected to the LAN 452 through a wired and/or wireless communication network interface or adapter 456. The adapter 456 can facilitate wired or wireless communication to the LAN 452, which can also comprise a wireless AP disposed thereon for communicating with the adapter 456.

When used in a WAN networking environment, the computer 402 can comprise a modem 458 or can be connected to a communications server on the WAN 454 or has other means for establishing communications over the WAN 454, such as by way of the Internet. The modem 458, which can be internal or external and a wired or wireless device, can be connected to the system bus 408 via the input device interface 442. In a networked environment, program modules depicted relative to the computer 402 or portions thereof, can be stored in the remote memory/storage device 450. It will be appreciated that the network connections shown are example and other means of establishing a communications link between the computers can be used.

The computer 402 can be operable to communicate with any wireless devices or entities operatively disposed in wireless communication, e.g., a printer, scanner, desktop and/or portable computer, portable data assistant, communications satellite, any piece of equipment or location associated with a wirelessly detectable tag (e.g., a kiosk, news stand, restroom), and telephone. This can comprise Wireless Fidelity (Wi-Fi) and BLUETOOTH® wireless technologies. Thus, the communication can be a predefined structure as with a conventional network or simply an ad hoc communication between at least two devices.

Wi-Fi can allow connection to the Internet from a couch at home, a bed in a hotel room or a conference room at work, without wires. Wi-Fi is a wireless technology similar to that used in a cell phone that enables such devices, e.g., computers, to send and receive data indoors and out; anywhere within the range of a base station. Wi-Fi networks use radio technologies called IEEE 802.11 (a, b, g, n, ac, ag, etc.) to provide secure, reliable, fast wireless connectivity. A Wi-Fi network can be used to connect computers to each other, to the Internet, and to wired networks (which can use IEEE 802.3 or Ethernet). Wi-Fi networks operate in the unlicensed 2.4 and 5 GHz radio bands for example or with products that contain both bands (dual band), so the networks can provide real-world performance similar to the basic 10BaseT wired Ethernet networks used in many offices.

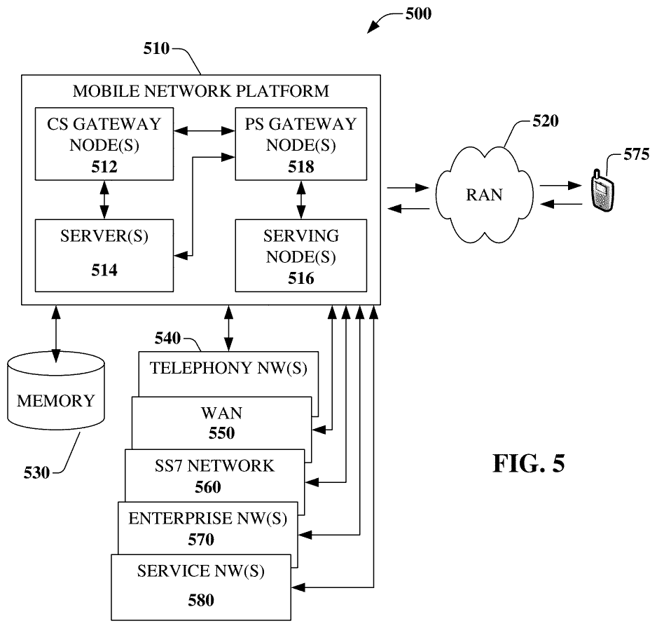

Turning now to FIG. 5, an embodiment 500 of a mobile network platform 510 is shown that is an example of network elements 150, 152, 154, 156, and/or VNEs 330, 332, 334, etc. For example, platform 510 can facilitate in whole or in part obtaining a first request for medical attention to be administered to a first user, wherein the first user is in possession of a first communication device that stores first data, transmitting an identifier associated with the first request, wherein the identifier is received by a second communication device associated with a second user, obtaining a second request from the second communication device, the second request including the identifier, based on the obtaining of the second request, generating a first key based on second data associated with the second user, and transmitting the first key to the second communication device such that the second communication device obtains a first portion of the first data from the first communication device by transmitting the first key to the first communication device. Platform 510 can facilitate in whole or in part transmitting an identifier associated with a first request for medical attention to be administered to a first user, wherein the first user is associated with a first user equipment that stores first data pertaining to a medical history of the first user, and wherein the identifier is received by a second user equipment associated with a second user and a third user equipment associated with a third user, obtaining a second request from the second user equipment, the second request including the identifier, based on the obtaining of the second request, generating a first key based on second data associated with the second user, wherein the first key grants the second user equipment access to a first portion of the first data, obtaining a third request from the third user equipment, the third request including the identifier, based on the obtaining of the third request, generating a second key based on third data associated with the third user, wherein the second key grants the third user equipment access to a second portion of the first data, the second portion of the first data being at least partially different from the first portion of the first data, transmitting the first key to the second user equipment, and transmitting the second key to the third user equipment. Platform 510 can facilitate in whole or in part transmitting, by a processing system including a processor, a request for attention, wherein the processing system stores first data pertaining to a first user, receiving, by the processing system and based on the transmitting of the request for attention, a key from a second processing system including a second processor, based on the receiving of the key, identifying, by the processing system, a first portion of the first data to be transmitted to the second processing system, wherein the first portion of the first data is less than an entirety of the first data, and based on the identifying of the first portion of the first data, transmitting, by the processing system, the first portion of the first data to the second processing system.

In one or more embodiments, the mobile network platform 510 can generate and receive signals transmitted and received by base stations or access points such as base station or access point 122. Generally, mobile network platform 510 can comprise components, e.g., nodes, gateways, interfaces, servers, or disparate platforms, that facilitate both packet-switched (PS) (e.g., internet protocol (IP), frame relay, asynchronous transfer mode (ATM)) and circuit-switched (CS) traffic (e.g., voice and data), as well as control generation for networked wireless telecommunication. As a non-limiting example, mobile network platform 510 can be included in telecommunications carrier networks, and can be considered carrier-side components as discussed elsewhere herein. Mobile network platform 510 comprises CS gateway node(s) 512 which can interface CS traffic received from legacy networks like telephony network(s) 540 (e.g., public switched telephone network (PSTN), or public land mobile network (PLMN)) or a signaling system #7 (SS7) network 560. CS gateway node(s) 512 can authorize and authenticate traffic (e.g., voice) arising from such networks. Additionally, CS gateway node(s) 512 can access mobility, or roaming, data generated through SS7 network 560; for instance, mobility data stored in a visited location register (VLR), which can reside in memory 530. Moreover, CS gateway node(s) 512 interfaces CS-based traffic and signaling and PS gateway node(s) 518. As an example, in a 3GPP UMTS network, CS gateway node(s) 512 can be realized at least in part in gateway GPRS support node(s) (GGSN). It should be appreciated that functionality and specific operation of CS gateway node(s) 512, PS gateway node(s) 518, and serving node(s) 516, is provided and dictated by radio technology(ies) utilized by mobile network platform 510 for telecommunication over a radio access network 520 with other devices, such as a radiotelephone 575.

In addition to receiving and processing CS-switched traffic and signaling, PS gateway node(s) 518 can authorize and authenticate PS-based data sessions with served mobile devices. Data sessions can comprise traffic, or content(s), exchanged with networks external to the mobile network platform 510, like wide area network(s) (WANs) 550, enterprise network(s) 570, and service network(s) 580, which can be embodied in local area network(s) (LANs), can also be interfaced with mobile network platform 510 through PS gateway node(s) 518. It is to be noted that WANs 550 and enterprise network(s) 570 can embody, at least in part, a service network(s) like IP multimedia subsystem (IMS). Based on radio technology layer(s) available in technology resource(s) or radio access network 520, PS gateway node(s) 518 can generate packet data protocol contexts when a data session is established; other data structures that facilitate routing of packetized data also can be generated. To that end, in an aspect, PS gateway node(s) 518 can comprise a tunnel interface (e.g., tunnel termination gateway (TTG) in 3GPP UMTS network(s) (not shown)) which can facilitate packetized communication with disparate wireless network(s), such as Wi-Fi networks.

In embodiment 500, mobile network platform 510 also comprises serving node(s) 516 that, based upon available radio technology layer(s) within technology resource(s) in the radio access network 520, convey the various packetized flows of data streams received through PS gateway node(s) 518. It is to be noted that for technology resource(s) that rely primarily on CS communication, server node(s) can deliver traffic without reliance on PS gateway node(s) 518; for example, server node(s) can embody at least in part a mobile switching center. As an example, in a 3GPP UMTS network, serving node(s) 516 can be embodied in serving GPRS support node(s) (SGSN).

For radio technologies that exploit packetized communication, server(s) 514 in mobile network platform 510 can execute numerous applications that can generate multiple disparate packetized data streams or flows, and manage (e.g., schedule, queue, format . . . ) such flows. Such application(s) can comprise add-on features to standard services (for example, provisioning, billing, customer support . . . ) provided by mobile network platform 510. Data streams (e.g., content(s) that are part of a voice call or data session) can be conveyed to PS gateway node(s) 518 for authorization/authentication and initiation of a data session, and to serving node(s) 516 for communication thereafter. In addition to application server, server(s) 514 can comprise utility server(s), a utility server can comprise a provisioning server, an operations and maintenance server, a security server that can implement at least in part a certificate authority and firewalls as well as other security mechanisms, and the like. In an aspect, security server(s) secure communication served through mobile network platform 510 to ensure network's operation and data integrity in addition to authorization and authentication procedures that CS gateway node(s) 512 and PS gateway node(s) 518 can enact. Moreover, provisioning server(s) can provision services from external network(s) like networks operated by a disparate service provider; for instance, WAN 550 or Global Positioning System (GPS) network(s) (not shown). Provisioning server(s) can also provision coverage through networks associated to mobile network platform 510 (e.g., deployed and operated by the same service provider), such as the distributed antennas networks shown in FIG. 1(s) that enhance wireless service coverage by providing more network coverage.

It is to be noted that server(s) 514 can comprise one or more processors configured to confer at least in part the functionality of mobile network platform 510. To that end, the one or more processor can execute code instructions stored in memory 530, for example. It is should be appreciated that server(s) 514 can comprise a content manager, which operates in substantially the same manner as described hereinbefore.

In example embodiment 500, memory 530 can store information related to operation of mobile network platform 510. Other operational information can comprise provisioning information of mobile devices served through mobile network platform 510, subscriber databases; application intelligence, pricing schemes, e.g., promotional rates, flat-rate programs, couponing campaigns; technical specification(s) consistent with telecommunication protocols for operation of disparate radio, or wireless, technology layers; and so forth. Memory 530 can also store information from at least one of telephony network(s) 540, WAN 550, SS7 network 560, or enterprise network(s) 570. In an aspect, memory 530 can be, for example, accessed as part of a data store component or as a remotely connected memory store.

In order to provide a context for the various aspects of the disclosed subject matter, FIG. 5, and the following discussion, are intended to provide a brief, general description of a suitable environment in which the various aspects of the disclosed subject matter can be implemented. While the subject matter has been described above in the general context of computer-executable instructions of a computer program that runs on a computer and/or computers, those skilled in the art will recognize that the disclosed subject matter also can be implemented in combination with other program modules. Generally, program modules comprise routines, programs, components, data structures, etc. that perform particular tasks and/or implement particular abstract data types.

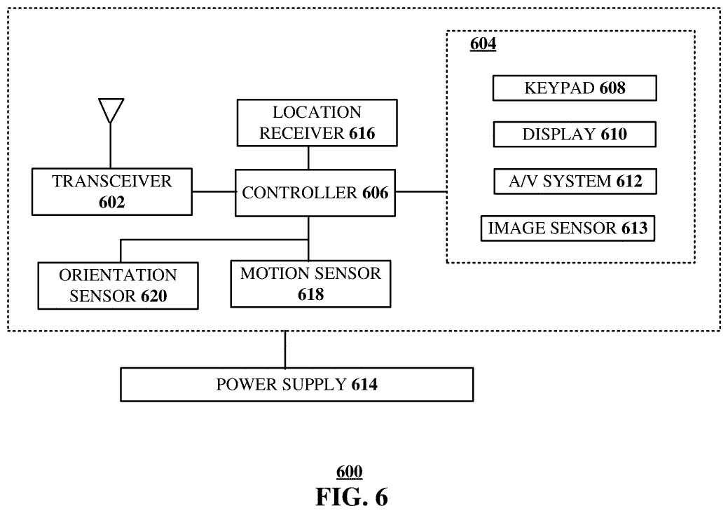

Turning now to FIG. 6, an illustrative embodiment of a communication device 600 is shown. The communication device 600 can serve as an illustrative embodiment of devices such as data terminals 114, mobile devices 124, vehicle 126, display devices 144 or other client devices for communication via either communications network 125. For example, computing device 600 can facilitate in whole or in part obtaining a first request for medical attention to be administered to a first user, wherein the first user is in possession of a first communication device that stores first data, transmitting an identifier associated with the first request, wherein the identifier is received by a second communication device associated with a second user, obtaining a second request from the second communication device, the second request including the identifier, based on the obtaining of the second request, generating a first key based on second data associated with the second user, and transmitting the first key to the second communication device such that the second communication device obtains a first portion of the first data from the first communication device by transmitting the first key to the first communication device. Computing device 600 can facilitate in whole or in part transmitting an identifier associated with a first request for medical attention to be administered to a first user, wherein the first user is associated with a first user equipment that stores first data pertaining to a medical history of the first user, and wherein the identifier is received by a second user equipment associated with a second user and a third user equipment associated with a third user, obtaining a second request from the second user equipment, the second request including the identifier, based on the obtaining of the second request, generating a first key based on second data associated with the second user, wherein the first key grants the second user equipment access to a first portion of the first data, obtaining a third request from the third user equipment, the third request including the identifier, based on the obtaining of the third request, generating a second key based on third data associated with the third user, wherein the second key grants the third user equipment access to a second portion of the first data, the second portion of the first data being at least partially different from the first portion of the first data, transmitting the first key to the second user equipment, and transmitting the second key to the third user equipment. Computing device 600 can facilitate in whole or in part transmitting, by a processing system including a processor, a request for attention, wherein the processing system stores first data pertaining to a first user, receiving, by the processing system and based on the transmitting of the request for attention, a key from a second processing system including a second processor, based on the receiving of the key, identifying, by the processing system, a first portion of the first data to be transmitted to the second processing system, wherein the first portion of the first data is less than an entirety of the first data, and based on the identifying of the first portion of the first data, transmitting, by the processing system, the first portion of the first data to the second processing system.

The communication device 600 can comprise a wireline and/or wireless transceiver 602 (herein transceiver 602), a user interface (UI) 604, a power supply 614, a location receiver 616, a motion sensor 618, an orientation sensor 620, and a controller 606 for managing operations thereof. The transceiver 602 can support short-range or long-range wireless access technologies such as Bluetooth®, ZigBee®, WiFi, DECT, or cellular communication technologies, just to mention a few (Bluetooth® and ZigBee® are trademarks registered by the Bluetooth® Special Interest Group and the ZigBee® Alliance, respectively). Cellular technologies can include, for example, CDMA-1X, UMTS/HSDPA, GSM/GPRS, TDMA/EDGE, EV/DO, WiMAX, SDR, LTE, as well as other next generation wireless communication technologies as they arise. The transceiver 602 can also be adapted to support circuit-switched wireline access technologies (such as PSTN), packet-switched wireline access technologies (such as TCP/IP, VoIP, etc.), and combinations thereof.

The UI 604 can include a depressible or touch-sensitive keypad 608 with a navigation mechanism such as a roller ball, a joystick, a mouse, or a navigation disk for manipulating operations of the communication device 600. The keypad 608 can be an integral part of a housing assembly of the communication device 600 or an independent device operably coupled thereto by a tethered wireline interface (such as a USB cable) or a wireless interface supporting for example Bluetooth®. The keypad 608 can represent a numeric keypad commonly used by phones, and/or a QWERTY keypad with alphanumeric keys. The UI 604 can further include a display 610 such as monochrome or color LCD (Liquid Crystal Display), OLED (Organic Light Emitting Diode) or other suitable display technology for conveying images to an end user of the communication device 600. In an embodiment where the display 610 is touch-sensitive, a portion or all of the keypad 608 can be presented by way of the display 610 with navigation features.

The display 610 can use touch screen technology to also serve as a user interface for detecting user input. As a touch screen display, the communication device 600 can be adapted to present a user interface having graphical user interface (GUI) elements that can be selected by a user with a touch of a finger. The display 610 can be equipped with capacitive, resistive or other forms of sensing technology to detect how much surface area of a user's finger has been placed on a portion of the touch screen display. This sensing information can be used to control the manipulation of the GUI elements or other functions of the user interface. The display 610 can be an integral part of the housing assembly of the communication device 600 or an independent device communicatively coupled thereto by a tethered wireline interface (such as a cable) or a wireless interface.

The UI 604 can also include an audio system 612 that utilizes audio technology for conveying low volume audio (such as audio heard in proximity of a human ear) and high volume audio (such as speakerphone for hands free operation). The audio system 612 can further include a microphone for receiving audible signals of an end user. The audio system 612 can also be used for voice recognition applications. The UI 604 can further include an image sensor 613 such as a charged coupled device (CCD) camera for capturing still or moving images.

The power supply 614 can utilize common power management technologies such as replaceable and rechargeable batteries, supply regulation technologies, and/or charging system technologies for supplying energy to the components of the communication device 600 to facilitate long-range or short-range portable communications. Alternatively, or in combination, the charging system can utilize external power sources such as DC power supplied over a physical interface such as a USB port or other suitable tethering technologies.

The location receiver 616 can utilize location technology such as a global positioning system (GPS) receiver capable of assisted GPS for identifying a location of the communication device 600 based on signals generated by a constellation of GPS satellites, which can be used for facilitating location services such as navigation. The motion sensor 618 can utilize motion sensing technology such as an accelerometer, a gyroscope, or other suitable motion sensing technology to detect motion of the communication device 600 in three-dimensional space. The orientation sensor 620 can utilize orientation sensing technology such as a magnetometer to detect the orientation of the communication device 600 (north, south, west, and east, as well as combined orientations in degrees, minutes, or other suitable orientation metrics).

The communication device 600 can use the transceiver 602 to also determine a proximity to a cellular, WiFi, Bluetooth®, or other wireless access points by sensing techniques such as utilizing a received signal strength indicator (RSSI) and/or signal time of arrival (TOA) or time of flight (TOF) measurements. The controller 606 can utilize computing technologies such as a microprocessor, a digital signal processor (DSP), programmable gate arrays, application specific integrated circuits, and/or a video processor with associated storage memory such as Flash, ROM, RAM, SRAM, DRAM or other storage technologies for executing computer instructions, controlling, and processing data supplied by the aforementioned components of the communication device 600.

Other components not shown in FIG. 6 can be used in one or more embodiments of the subject disclosure. For instance, the communication device 600 can include a slot for adding or removing an identity module such as a Subscriber Identity Module (SIM) card or Universal Integrated Circuit Card (UICC). SIM or UICC cards can be used for identifying subscriber services, executing programs, storing subscriber data, and so on.

The terms “first,” “second,” “third,” and so forth, as used in the claims, unless otherwise clear by context, is for clarity only and doesn't otherwise indicate or imply any order in time. For instance, “a first determination,” “a second determination,” and “a third determination,” does not indicate or imply that the first determination is to be made before the second determination, or vice versa, etc.

In the subject specification, terms such as “store,” “storage,” “data store,” data storage,” “database,” and substantially any other information storage component relevant to operation and functionality of a component, refer to “memory components,” or entities embodied in a “memory” or components comprising the memory. It will be appreciated that the memory components described herein can be either volatile memory or nonvolatile memory, or can comprise both volatile and nonvolatile memory, by way of illustration, and not limitation, volatile memory, non-volatile memory, disk storage, and memory storage. Further, nonvolatile memory can be included in read only memory (ROM), programmable ROM (PROM), electrically programmable ROM (EPROM), electrically erasable ROM (EEPROM), or flash memory. Volatile memory can comprise random access memory (RAM), which acts as external cache memory. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), and direct Rambus RAM (DRRAM). Additionally, the disclosed memory components of systems or methods herein are intended to comprise, without being limited to comprising, these and any other suitable types of memory.

Moreover, it will be noted that the disclosed subject matter can be practiced with other computer system configurations, comprising single-processor or multiprocessor computer systems, mini-computing devices, mainframe computers, as well as personal computers, hand-held computing devices (e.g., PDA, phone, smartphone, watch, tablet computers, netbook computers, etc.), microprocessor-based or programmable consumer or industrial electronics, and the like. The illustrated aspects can also be practiced in distributed computing environments where tasks are performed by remote processing devices that are linked through a communications network; however, some if not all aspects of the subject disclosure can be practiced on stand-alone computers. In a distributed computing environment, program modules can be located in both local and remote memory storage devices.

In one or more embodiments, information regarding use of services can be generated including services being accessed, media consumption history, user preferences, and so forth. This information can be obtained by various methods including user input, detecting types of communications (e.g., video content vs. audio content), analysis of content streams, sampling, and so forth. The generating, obtaining and/or monitoring of this information can be responsive to an authorization provided by the user. In one or more embodiments, an analysis of data can be subject to authorization from user(s) associated with the data, such as an opt-in, an opt-out, acknowledgement requirements, notifications, selective authorization based on types of data, and so forth.

Some of the embodiments described herein can also employ artificial intelligence (AI) to facilitate automating one or more features described herein. The embodiments (e.g., in connection with automatically identifying acquired cell sites that provide a maximum value/benefit after addition to an existing communication network) can employ various AI-based schemes for carrying out various embodiments thereof. Moreover, the classifier can be employed to determine a ranking or priority of each cell site of the acquired network. A classifier is a function that maps an input attribute vector, x=(x1, x2, x3, x4, . . . , xn), to a confidence that the input belongs to a class, that is, f(x)=confidence (class). Such classification can employ a probabilistic and/or statistical-based analysis (e.g., factoring into the analysis utilities and costs) to determine or infer an action that a user desires to be automatically performed. A support vector machine (SVM) is an example of a classifier that can be employed. The SVM operates by finding a hypersurface in the space of possible inputs, which the hypersurface attempts to split the triggering criteria from the non-triggering events. Intuitively, this makes the classification correct for testing data that is near, but not identical to training data. Other directed and undirected model classification approaches comprise, e.g., naïve Bayes, Bayesian networks, decision trees, neural networks, fuzzy logic models, and probabilistic classification models providing different patterns of independence can be employed. Classification as used herein also is inclusive of statistical regression that is utilized to develop models of priority.

As will be readily appreciated, one or more of the embodiments can employ classifiers that are explicitly trained (e.g., via a generic training data) as well as implicitly trained (e.g., via observing UE behavior, operator preferences, historical information, receiving extrinsic information). For example, SVMs can be configured via a learning or training phase within a classifier constructor and feature selection module. Thus, the classifier(s) can be used to automatically learn and perform a number of functions, including but not limited to determining according to predetermined criteria which of the acquired cell sites will benefit a maximum number of subscribers and/or which of the acquired cell sites will add minimum value to the existing communication network coverage, etc.

As used in some contexts in this application, in some embodiments, the terms “component,” “system” and the like are intended to refer to, or comprise, a computer-related entity or an entity related to an operational apparatus with one or more specific functionalities, wherein the entity can be either hardware, a combination of hardware and software, software, or software in execution. As an example, a component may be, but is not limited to being, a process running on a processor, a processor, an object, an executable, a thread of execution, computer-executable instructions, a program, and/or a computer. By way of illustration and not limitation, both an application running on a server and the server can be a component. One or more components may reside within a process and/or thread of execution and a component may be localized on one computer and/or distributed between two or more computers. In addition, these components can execute from various computer readable media having various data structures stored thereon. The components may communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor, wherein the processor can be internal or external to the apparatus and executes at least a part of the software or firmware application. As yet another example, a component can be an apparatus that provides specific functionality through electronic components without mechanical parts, the electronic components can comprise a processor therein to execute software or firmware that confers at least in part the functionality of the electronic components. While various components have been illustrated as separate components, it will be appreciated that multiple components can be implemented as a single component, or a single component can be implemented as multiple components, without departing from example embodiments.

Further, the various embodiments can be implemented as a method, apparatus or article of manufacture using standard programming and/or engineering techniques to produce software, firmware, hardware or any combination thereof to control a computer to implement the disclosed subject matter. The term “article of manufacture” as used herein is intended to encompass a computer program accessible from any computer-readable device or computer-readable storage/communications media. For example, computer readable storage media can include, but are not limited to, magnetic storage devices (e.g., hard disk, floppy disk, magnetic strips), optical disks (e.g., compact disk (CD), digital versatile disk (DVD)), smart cards, and flash memory devices (e.g., card, stick, key drive). Of course, those skilled in the art will recognize many modifications can be made to this configuration without departing from the scope or spirit of the various embodiments.

In addition, the words “example” and “exemplary” are used herein to mean serving as an instance or illustration. Any embodiment or design described herein as “example” or “exemplary” is not necessarily to be construed as preferred or advantageous over other embodiments or designs. Rather, use of the word example or exemplary is intended to present concepts in a concrete fashion. As used in this application, the term “or” is intended to mean an inclusive “or” rather than an exclusive “or”. That is, unless specified otherwise or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Moreover, terms such as “user equipment,” “mobile station,” “mobile,” subscriber station,” “access terminal,” “terminal,” “handset,” “mobile device” (and/or terms representing similar terminology) can refer to a wireless device utilized by a subscriber or user of a wireless communication service to receive or convey data, control, voice, video, sound, gaming or substantially any data-stream or signaling-stream. The foregoing terms are utilized interchangeably herein and with reference to the related drawings.

Furthermore, the terms “user,” “subscriber,” “customer,” “consumer” and the like are employed interchangeably throughout, unless context warrants particular distinctions among the terms. It should be appreciated that such terms can refer to human entities or automated components supported through artificial intelligence (e.g., a capacity to make inference based, at least, on complex mathematical formalisms), which can provide simulated vision, sound recognition and so forth.

As employed herein, the term “processor” can refer to substantially any computing processing unit or device comprising, but not limited to comprising, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components or any combination thereof designed to perform the functions described herein. Processors can exploit nano-scale architectures such as, but not limited to, molecular and quantum-dot based transistors, switches and gates, in order to optimize space usage or enhance performance of user equipment. A processor can also be implemented as a combination of computing processing units.

As used herein, terms such as “data storage,” data storage,” “database,” and substantially any other information storage component relevant to operation and functionality of a component, refer to “memory components,” or entities embodied in a “memory” or components comprising the memory. It will be appreciated that the memory components or computer-readable storage media, described herein can be either volatile memory or nonvolatile memory or can include both volatile and nonvolatile memory.

What has been described above includes mere examples of various embodiments. It is, of course, not possible to describe every conceivable combination of components or methodologies for purposes of describing these examples, but one of ordinary skill in the art can recognize that many further combinations and permutations of the present embodiments are possible. Accordingly, the embodiments disclosed and/or claimed herein are intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

In addition, a flow diagram may include a “start” and/or “continue” indication. The “start” and “continue” indications reflect that the steps presented can optionally be incorporated in or otherwise used in conjunction with other routines. In this context, “start” indicates the beginning of the first step presented and may be preceded by other activities not specifically shown. Further, the “continue” indication reflects that the steps presented may be performed multiple times and/or may be succeeded by other activities not specifically shown. Further, while a flow diagram indicates a particular ordering of steps, other orderings are likewise possible provided that the principles of causality are maintained.

As may also be used herein, the term(s) “operably coupled to”, “coupled to”, and/or “coupling” includes direct coupling between items and/or indirect coupling between items via one or more intervening items. Such items and intervening items include, but are not limited to, junctions, communication paths, components, circuit elements, circuits, functional blocks, and/or devices. As an example of indirect coupling, a signal conveyed from a first item to a second item may be modified by one or more intervening items by modifying the form, nature or format of information in a signal, while one or more elements of the information in the signal are nevertheless conveyed in a manner than can be recognized by the second item. In a further example of indirect coupling, an action in a first item can cause a reaction on the second item, as a result of actions and/or reactions in one or more intervening items.

Although specific embodiments have been illustrated and described herein, it should be appreciated that any arrangement which achieves the same or similar purpose may be substituted for the embodiments described or shown by the subject disclosure. The subject disclosure is intended to cover any and all adaptations or variations of various embodiments. Combinations of the above embodiments, and other embodiments not specifically described herein, can be used in the subject disclosure. For instance, one or more features from one or more embodiments can be combined with one or more features of one or more other embodiments. In one or more embodiments, features that are positively recited can also be negatively recited and excluded from the embodiment with or without replacement by another structural and/or functional feature. The steps or functions described with respect to the embodiments of the subject disclosure can be performed in any order. The steps or functions described with respect to the embodiments of the subject disclosure can be performed alone or in combination with other steps or functions of the subject disclosure, as well as from other embodiments or from other steps that have not been described in the subject disclosure. Further, more than or less than all of the features described with respect to an embodiment can also be utilized.

What is claimed is:

1. A device, comprising:
a processing system including a processor; and
a memory that stores executable instructions that, when executed by the processing system, facilitate performance of operations, the operations comprising:
obtaining a first request for medical attention to be administered to a first user, wherein the first user is in possession of a first communication device that stores first data;
transmitting an identifier associated with the first request, wherein the identifier is received by a second communication device associated with a second user;
obtaining a second request from the second communication device, the second request including the identifier;
based on the obtaining of the second request, generating a first key based on second data associated with the second user; and
transmitting the first key to the second communication device such that the second communication device obtains a first portion of the first data from the first communication device by transmitting the first key to the first communication device.

2. The device of claim 1, wherein the second communication device analyzes the first portion of the first data and generates a directive that commands the second user to perform a task as part of administering the medical attention.

3. The device of claim 1, wherein the identifier is received by a third communication device associated with a third user.

4. The device of claim 3, wherein the operations further comprise:
obtaining a third request from the third communication device, the third request including the identifier; and
based on the obtaining of the third request, generating a second key based on third data associated with the third user.

5. The device of claim 4, wherein the third data is different from the second data, and wherein the second key is different from the first key.

6. The device of claim 4, wherein the operations further comprise:
transmitting the second key to the third communication device such that the third communication device obtains a second portion of the first data from the first communication device by transmitting the second key to the first communication device.

7. The device of claim 6, wherein the first portion of the first data and the second portion of the first data are at least partially different from one another.

8. The device of claim 1, wherein the obtaining of the first request comprises obtaining the first request from the first communication device.

9. The device of claim 1, wherein the first request is initiated at the first communication device based on an output of a sensor that is associated with the first communication device.

10. The device of claim 1, wherein the first request is initiated at the first communication device based on a user input manually generated by the first user at the first communication device.

11. The device of claim 1, wherein the first request is initiated at a third communication device associated with a third user.

12. The device of claim 1, wherein the operations further comprise:
identifying a first location of the first communication device;
identifying a second location of the second communication device;
comparing the first location to the second location to identify a distance between the first communication device and the second communication device; and
determining that the distance is less than a threshold,
wherein the transmitting of the identifier associated with the first request comprises directing the identifier to the second communication device based on the determining indicating that the distance is less than the threshold.

13. The device of claim 1, wherein the operations further comprise:
identifying a first location of the first communication device;
identifying a second location of the second communication device;
comparing the first location to the second location to identify a first distance between the first communication device and the second communication device;
identifying a third location of a third communication device associated with a third user;
comparing the first location to the third location to identify a second distance between the first communication device and the third communication device; and
determining that the first distance is less than the second distance,
wherein the transmitting of the identifier associated with the first request comprises directing the identifier to the second communication device based on the determining indicating that the first distance is less than the second distance.

14. The device of claim 1, wherein the first key, an application executed by the second communication device, or a combination thereof, prohibits the second communication device from: storing the first portion of the first data in a memory device of the second communication device, transmitting the first portion of the first data, editing the first portion of the first data, or any combination thereof.

15. The device of claim 1, wherein the operations further comprise:
obtaining third data from the first communication device, the third data associated with the administration of the medical attention.

16. The device of claim 15, wherein the first communication device obtains the third data from the second communication device.

17. A non-transitory machine-readable medium, comprising executable instructions that, when executed by a processing system including a processor, facilitate performance of operations, the operations comprising:
transmitting an identifier associated with a first request for medical attention to be administered to a first user, wherein the first user is associated with a first user equipment that stores first data pertaining to a medical history of the first user, and wherein the identifier is received by a second user equipment associated with a second user and a third user equipment associated with a third user;
obtaining a second request from the second user equipment, the second request including the identifier;
based on the obtaining of the second request, generating a first key based on second data associated with the second user, wherein the first key grants the second user equipment access to a first portion of the first data;
obtaining a third request from the third user equipment, the third request including the identifier;
based on the obtaining of the third request, generating a second key based on third data associated with the third user, wherein the second key grants the third user equipment access to a second portion of the first data, the second portion of the first data being at least partially different from the first portion of the first data;
transmitting the first key to the second user equipment; and
transmitting the second key to the third user equipment.

18. The non-transitory machine-readable medium of claim 17, wherein the first portion of the first data and the second portion of the first data each omit: a name of the first user, a residential address of the first user, and a birthday of the first user.

19. A method, comprising:
transmitting, by a processing system including a processor, a request for attention, wherein the processing system stores first data pertaining to a first user;
receiving, by the processing system and based on the transmitting of the request for attention, a key from a second processing system including a second processor;
based on the receiving of the key, identifying, by the processing system, a first portion of the first data to be transmitted to the second processing system, wherein the first portion of the first data is less than an entirety of the first data; and
based on the identifying of the first portion of the first data, transmitting, by the processing system, the first portion of the first data to the second processing system.

20. The method of claim 19, further comprising:
receiving, by the processing system, an identifier associated with the second processing system,
wherein the transmitting of the first portion of the first data to the second processing system is based on the receiving of the identifier.
